(19) United States
(12) Patent Application Publication
(10) Pub. No.: US 2008/0200762 A1
(43) Pub. Date: Aug. 21, 2008

(54) Flexible endoscope shapelock

(76) Inventors: Michael J. Stokes, Cincinnati, OH (US); David Stefanchik, Morrow, OH (US)

Correspondence Address: KIRKPATRICK & LOCKHART PRESTON GATES ELLIS LLP, 535 SMITHFIELD STREET, PITTSBURGH, PA, 15222, US

(21) Appl. No.: 11/707,831

(22) Filed: Feb. 16, 2007

(51) Int. Cl. A61B 1/12

(52) U.S. Cl. 600/139; 600/114

(57) ABSTRACT

A medical apparatus includes a flexible sheath adapted to receive an endoscope. A first flexible rail is formed on the flexible sheath. The first flexible rail extends longitudinally along the length of the flexible sheath. A first rigidizable guide member includes a first track channel adapted to receive the first flexible rail. The first rigidizable guide member is adapted to slideably move along the first flexible rail and the first track channel.

### BACKGROUND

The embodiments are related generally to medical devices and more particularly to devices and methods useful in endoscopic procedures.

Minimally invasive procedures are desirable because such procedures can reduce pain and provide relatively quick recovery times as compared with conventional open medical procedures. Many minimally invasive procedures are performed with an endoscope (including without limitation laparoscopes). Such procedures permit a physician to position, manipulate, and view medical instruments and accessories inside the patient through a small access opening in the patient's body. Laparoscopy is a term used to describe such an “endosurgical” approach using an endoscope (often a rigid laparoscope). In this type of procedure, accessory devices are often inserted into a patient through trocars placed through the body wall.

Still less invasive treatments include those that are performed through insertion of an endoscope through a natural body orifice to a treatment region. Examples of this approach include, but are not limited to, cystoscopy, hysteroscopy, esophagogastroduodenoscopy, and colonoscopy. Many of these procedures employ the use of a flexible endoscope during the procedure. Flexible endoscopes often have a flexible, steerable articulating section near the distal end that can be controlled by the user by utilizing controls at the proximal end. Minimally invasive therapeutic procedures to treat diseased tissue by introducing medical instruments to a tissue treatment region through a natural opening of the patient are known as Natural Orifice Translumenal Endoscopic Surgery (NOTES)™.

Some flexible endoscopes are relatively small (1 mm to 3 mm in diameter), and may have no integral accessory channel (also called biopsy channels or working channels). Other flexible endoscopes, including gastroscopes and colonoscopes, have integral working channels having a diameter of about 2.0 to 3.5 mm for the purpose of introducing and removing medical devices and other accessory devices to perform diagnosis or therapy within the patient. As a result, the accessory devices used by a physician can be limited in size by the diameter of the accessory channel of the scope used. Additionally, the physician may be limited to a single accessory device when using the standard endoscope having one working channel.

Certain specialized endoscopes are available, such as large working channel endoscopes having a working channel of 5 mm in diameter, which can be used to pass relatively large accessories, or to provide capability to suction large blood clots. Other specialized endoscopes include those having two working channels. One disadvantages of such large diameter/multiple working channel endoscopes can be that such devices can be relatively expensive. Further, such large diameter/multiple working channel endoscopes can have an outer diameter that makes the endoscope relatively stiff, or otherwise difficult to intubate.

Inserting an endoscope through a natural body orifice to a tissue treatment region requires the ability to access the peritoneal cavity in various locations and angular positions. Current flexible endoscopes only allow for the distal portion of the endoscope to be maneuverable or lockable into a position. For example, in a transgastric choly procedure the endoscope can only approach the gallbladder from the level of the gastrostomy. Similar limitations exist with a transcolonic approach. In addition, for colonoscopies, it is often painful for the patient as the flexible endoscope is maneuvered past the splenic and the hepatic flexures. This may be due to the fact that only a few inches (approximately five inches) at the distal end of the endoscope are positionable and the rest of the endoscope is flexed by interacting with the colon.

There is a need for improved medical instruments to locate flexible endoscopes in within a patient in various locations and angular positions. There is a need to introduce these instruments in a natural opening of the patient.

### SUMMARY

In one general aspect, the various embodiments are directed to a medical apparatus. In one embodiment, the medical apparatus comprises a flexible sheath adapted to receive an endoscope. A first flexible rail is formed on the flexible sheath. The first flexible rail extends longitudinally along the length of the flexible sheath. A first rigidizable guide member comprises a first track channel adapted to receive the first flexible rail. The first rigidizable guide member is adapted to slideably move along the first flexible rail and the first track channel.

### FIGURES

The novel features of the various embodiments are set forth with particularity in the appended claims. The various embodiments, however, both as to organization and methods of operation may best be understood by reference to the following description, taken in conjunction with the accompanying drawings as follows.

FIG. 1 illustrates one embodiment of a medical apparatus.

FIG. 2 illustrates a partial sectional view of one embodiment of a rigidizable guide member taken along the longitudinal axis with a tension wire extending through a central bore.

FIG. 3A illustrates a partial sectional view of one embodiment of a rigidizable guide member taken along the longitudinal axis.

FIG. 3B is an enlargement of one embodiment of a state-change material that may be introduced into the central bore for the purpose of rigidizing the rigidizable guide member.

FIG. 4A illustrates a partial sectional view of one embodiment of a rigidizable guide member taken along the longitudinal axis.

FIG. 4B illustrates a partial sectional view of one embodiment of a rigidizable guide member taken along the longitudinal axis.

FIG. 5A is an end view of one embodiment of the medical apparatus shown in FIG. 1.

FIG. 5B is an end view of one embodiment of a medical apparatus.

FIGS. 6A-E illustrate one embodiment of a method of employing a medical apparatus comprising the first and second rigidizable guide members to advance and maneuver the endoscope into a natural hollow body organ of a patient having a tortuous and unsupported anatomy.

FIG. 7 shows one embodiment of the medical apparatus inserted into a hollow body organ or a natural opening of a patient.

### DESCRIPTION

The various embodiments described herein are directed to medical devices and more particularly to devices and methods useful in minimally invasive endoscopic procedures. The various embodiments provide methods and devices useful with various medical procedures, including without limitation methods and devices useful with endoscopes, methods and devices employed through naturally occurring body orifices, and methods and devices related to placement of feeding tubes. For example, in one embodiment, the medical device can be used to quickly and consistently place an endoscope in a desired location such as in the stomach or the jejunum. In various embodiments, the medical device may be employed to comfortably insert an endoscope through a natural body orifice to a treatment region of a patient through the peritoneal cavity in various locations and angular positions. Embodiments of the medical device reduce pain and discomfort to the patient as the endoscope is maneuvered inside the patient by making the endoscope positionable and flexible as it is advanced inside the patient. These and other embodiments are now illustrated and described with reference to the following figures.

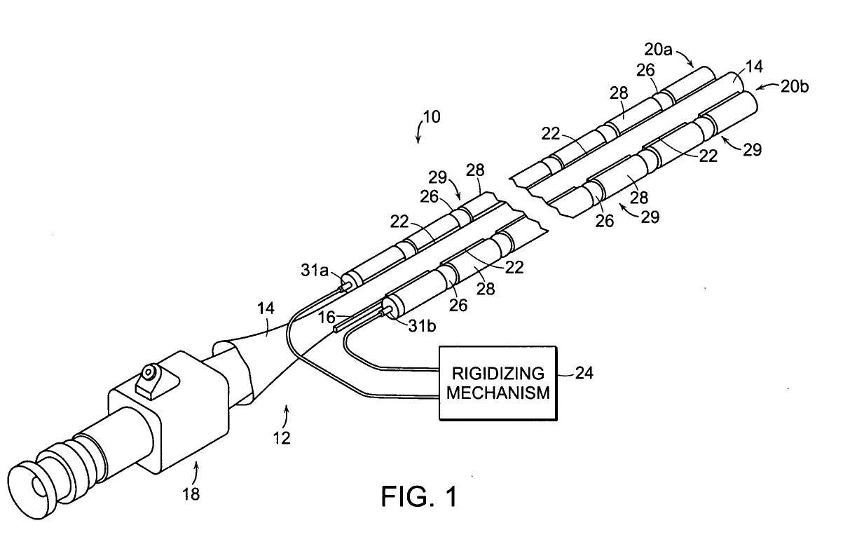

FIG. 1 illustrates one embodiment of a medical apparatus 10. The medical apparatus 10 comprises a handle 12, a flexible sheath 14 extending from the handle 12, and a flexible rail 16 disposed on the sheath 14. The flexible rail 16 comprises a web and is supported by the flexible sheath 14. The flexible sheath 14 can be joined to the flexible rail 16 by any suitable joining methods, such as ultrasonic welding. A cross-section of the flexible rail 16 and web defines a general “T” configuration. The flexible rail 16 can be a generally continuous, unitary piece of material which extends longitudinally along the length of the flexible sheath 14. The handle 12 and the flexible sheath 14 can be adapted to receive an endoscope 18 therethrough. In one embodiment, the flexible sheath and the flexible rail 16 may be formed integrally as a unitary member. The endoscope 18 comprises a substantially flexible shaft. A first normally flexible rigidizable guide member 20a and a second normally flexible rigidizable guide member 20b comprise a track channel 22 to slideably receive the flexible rail 16. The track channel 22 is supported by the rigidizable guide members 20a,b. By rigidizable it is meant that the guide members 20a,b may be rendered incapable of or resistant to bending and/or incapable of compromise or flexibility. A cross-section of the track channel 22 defines a general “C” configuration. The first and second rigidizable guide members 20a,b are coupled to a rigidizing mechanism 24. The rigidizing mechanism 24 may be any device capable of rendering the rigidizable guide members 20a,b rigid or inflexible. In various embodiments, the rigidizing mechanism 24 may be a wire tensioner, a vacuum pump, a combination of both, and/or other devices suitable to render the rigidizable guide members 20a,b rigid upon actuation. The rigidizing mechanism 24 may be disposed within the handle 12 or may be located remotely therefrom. In embodiments wherein the rigidizing mechanism 24 is a vacuum pump, the rigidizing mechanism 24 may be actuated or controlled by controls disposed on the handle 12.

It will be appreciated that the terms “proximal” and “distal” are used herein with reference to a clinician gripping the handle 12 of the instrument 10. Thus, the flexible portion of the endoscope 18 is distal with respect to the more proximal handle 12. It will be further appreciated that, for convenience and clarity, any spatial terms used herein with respect to the drawings. However, surgical instruments are used in many orientations and positions, and these terms are not intended to be limiting and absolute.

The endoscope 18 can be any commercially available endoscope, such as a gastroscope or colonoscope having an articulating distal section, including a viewing element and a working channel at the distal end thereof. Any suitable endoscope, including without limitation gastroscopes and pediatric colonscopes can be used with various embodiments of the medical apparatus 10. Suitable endoscopes for use with the present invention include, without limitation, model PCF100, PCF130L, PCF140L, or PCF160AL endoscopes manufactured by Olympus Corporation of Japan. The handle 12 and the flexible sheath 14 can be sized and adapted to receive various diameter endoscopes, such as, but not limited to, endoscopes having a diameter from about 9 mm to about 14 mm.

To introduce the endoscope 18 with the medical apparatus 10 into a patient, the operator may start with a clean dry endoscope. The flexible sheath 14 is preferably formed of a thin, light weight, drapable polymeric film material which can be relatively soft and elastically extensible, and which has substantially no torsional stiffness and no torsional load carrying capability. By “drapable” it is meant that the sheath does not maintain a circular or other regular cross-sectional shape in the absence of an internal structure (such as an endoscope) supporting the flexible sheath 14.

In one embodiment, the flexible sheath 14 can be formed of a material having an elastic modulus of less than about 20 ksi, more particularly less than about 15 ksi, still more particularly less than about 10 ksi, and even more particularly less than about 7 ksi. The flexible sheath 14 can be formed of a material having a yield strength of less than about 500 psi, more particularly less than about 300 psi, still more particularly less than about 200 psi, and still more particularly less than about 125 psi. In one embodiment, the flexible sheath 14 can be formed of a material having a yield strength of between about 90 psi and about 120 psi. The elastic modulus and yield strength can be determined as an average of five or more measurements, and can be determined using ASTM test #D882 (Standard Test Methods for Tensile Properties of Thin Plastic Sheeting) using a gage length of 4.0 inch, a gage width of 1.0 inch, a test thickness equal to the thickness of the film (e.g., about 0.005 inch), and a test machine speed of 0.4 in/minute. In one embodiment, the flexible sheath 14 can be formed of a film have a modulus of less than about 7 ksi, a yield strength of less than about 125 psi, and a tensile strength at break (measured according to ASTM D 638) of at least about 1 MPa (mega Pascal), more particularly at least about 5 Mpa, and still more particularly about 10 Mpa or greater. The flexible sheath 14 can be formed of a film having a tensile elongation (measured using ASTM D 638) of at least about 200 percent, more particularly at least about 500 percent, and still more particularly about 800 percent or more. The modulus, yield strength, tensile strength, and elongation are determined as mean of at least five measurements.

In some embodiments, it may be desirable that the flexible sheath 14 can be inserted over the insertion length of the endoscope 18 without use of a lubricant. In one embodiment, the flexible sheath 14 can have a non-smooth, textured inner surface that prevents the inner surface of the flexible sheath 14 from “sticking” to the outer surface of the insertion portion of the endoscope 18. The textured inner surface can also aid in gripping the endoscope 18 through the flexible sheath 14, such as for example if it is desired to rotate the flexible sheath 14 and the endoscope 18 together. The inner surface can be textured and the outer surface can be generally smooth, or both the inner and outer surfaces may be textured. The inner surface of the flexible sheath 14 may have the same texture as the outside surface, be relatively more textured than the outer surface, or be relatively less textured than the outside surface. Additional description of the flexible sheath 14 may be found in United States Patent Publication No. US 2006/0258907 titled “Track for Medical Devices” to Stefanchik et al., which is incorporated herein by reference in its entirety.

The medical apparatus 10 comprises rigidizable guide members 20a,b positioned along a longitudinal axis on either side of the endoscope 18. Each rigidizable guide member 20a,b may be advanced or retracted independently of each over a length of an adjustable portion of the endoscope 18, such as, for example, the steerable articulating section near the distal end of the endoscope 18. The steerable, articulating, or adjustable portion of the endoscope 18 is usually the distal five or six inches portion of the endoscope 18. Radially, the rigidizable guide members 20a,b may be coupled to the endoscope 18 by way of an endorail type connection. In the embodiment illustrated in FIG. 1, the endorail connection is formed of the flexible rail 16 having a general “T” configuration and the corresponding track channel 22 having a general “C” configuration, wherein the track channel 22 can be slideably advanced and retracted along the longitudinal length of the flexible rail 16. In various other embodiments, the flexible sheath 14 may be formed with a track channel having a general “C” configuration and the rigidizable guide members 20a,b may be formed with a flexible rail having a general “T” configuration so as to form another embodiment of an endorail connection. The illustrated embodiments are not limited in this context.

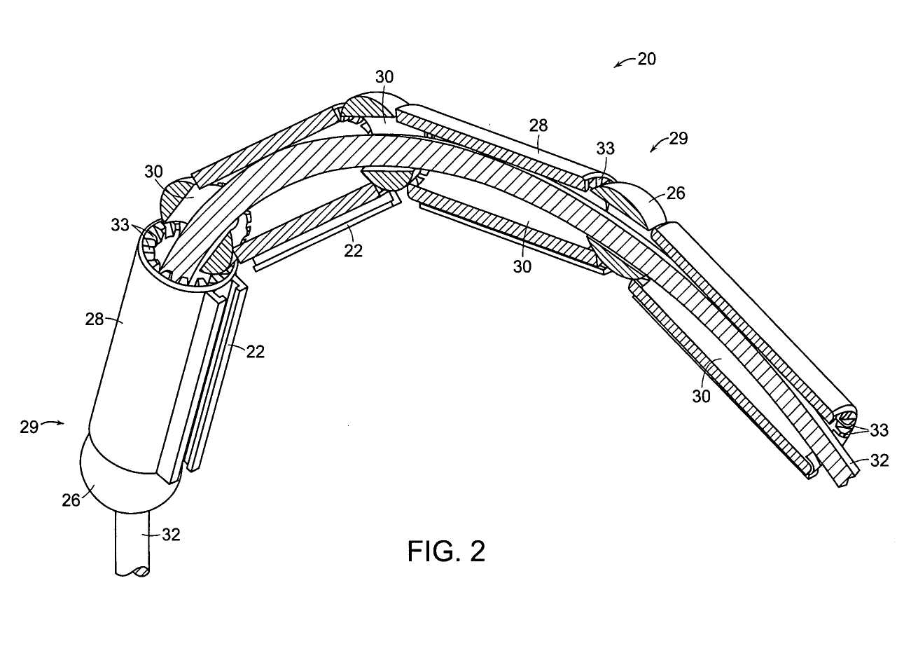

Each rigidizable guide member 20a,b also comprises a central bore 30 (FIG. 2) defining a channel for receiving rigidizing components. A rigidizing component may be introduced in the central bore 30. A rigidizing component is any device or material suitable to render the rigidizable guide members 20a,b rigid upon actuation of the rigidizing mechanism 24. In the rigid or inflexible mode, the rigidizable guide members 20a,b act as a guide or track to support the movement of each other and/or the endoscope 18. Flexibility may be restored when the rigidizing component is deactuated or the rigidizing force is removed. This process may be repeated as necessary. In one embodiment, the rigidizing component may comprise one or more tensioning wires to apply a clamping force on the rigidizable guide members 20a,b to render them rigid and inflexible. In another embodiment, the rigidizing component may comprise a state-change material disposed in the channel formed by the central bore 30 that becomes rigid when a vacuum is applied to vacuum ports 31a,b. In various other embodiments, the rigidizing component may comprise a combination of tensioning wires and the state-change material and thus may employ a combination of tensioning force and vacuum to render the rigidizable guide members 20a,b rigid. When the tensioning force or vacuum is released, the rigidizable guide members 20a,b return to their normally flexible state. In one embodiment, a flexible membrane (e.g., a sheath) may be provided over the rigidizable guide members 20a,b. Among other functions, the flexible membrane may assist when a vacuum is applied to the rigidizable guide members 20a,b to actuate the state-change material. In other embodiments, the flexible membrane may function as a protective cover for the rigidizable guide members 20a,b when located inside a natural body orifice of the patient. Any of the tensioning components may be operated by the rigidizing mechanism 24, which is a general mechanism adapted and configured to apply a suitable force necessary to actuate the rigidizing components. The embodiments, however, should not be limited in this context.

Embodiments of the rigidizable guide members 20a,b may be formed in various shapes, sizes, and materials. In one embodiment, rigidizable guide members may be formed with helical wires (e.g., coil spring). The highly flexible sheath 14 or a flexible membrane may be provided over such rigidizable guide members. The rigidizable guide members comprise a central bore that may be filled with biocompatible state-change material to render them rigid when a vacuum is applied. In another embodiment, the rigidizable guide members may be formed by connecting multiple cylindrical elements end-to-end held together by the highly flexible sheath 14 or the flexible membrane. The cylindrical elements provide radial stiffness. The central bore or channel of such rigidizable guide members may be filled with the biocompatible state-change material to render them rigid when a vacuum is applied. A combination of tension wires may be added to provide additional rigidizing capability.

In the embodiment illustrated in FIG. 1, the rigidizable guide members 20a,b may be formed with multiple assemblies 29 each comprising a ball 26 and a socket 28 and defining a central bore 30 (FIG. 2) therethrough. The ball 26 may be any spherical bead or element that may be insertable in a cylindrical sleeve such as the socket 26, such that in cooperation, the multiple ball 26 and socket 28 assemblies 29 render the rigidizable guide members 20a,b flexible in their normal state. The central bore 30 may be adapted to receive state-change material, one or more tension wires 32, or a combination thereof, to render the rigidizable guide members 20a,b rigid whenever the rigidizing mechanism 24 is actuated by an operator or another device. The ball 26 and socket 28 assemblies 29 may comprise a congruent pattern to provide additional locking force, and hence, additional rigidness.

FIG. 2 illustrates a partial sectional view of one embodiment of a rigidizable guide member 20 taken along the longitudinal axis with a tension wire 32 extending through a central bore 30. The rigidizable guide member 20 may be either the rigidizable guide member 20a or 20b shown in FIG. 1. The rigidizable guide member 20 comprises a continuous length assemblies 29 each comprising the nestable ball 26 and socket 28 components. In one embodiment, the ball 26 may be located (e.g., pressed) into and partially inserted into the socket 28 such that the ball 26 and socket 28 can rotate freely relative to each other and the ball 26 is retained within the socket 28. In one embodiment, the socket 28 may comprise projections 33 extending radially and inwardly and configured to engage and compress the surface of the ball 26. In one embodiment, the track channel 22 may be formed integrally with the socket 28 as a unitary piece. In other embodiments, the track channel 22 may be formed separately and attached to the socket 28 in any suitable manner (e.g., weld, adhesive). The track channel 22 is adapted and configured to slideably receive the flexible rail 16 to form the endorail connection. The track channel 22 is supported by the socket 28 portions of the rigidizable guide member 20 along the entire length of the rigidizable guide member 20. Accordingly, the track channel 22 is segmented along the length of the rigidizable guide member 20. A cross-section of the track channel 22 defines a general “C” configuration to receive the general “T” cross-sectional configuration of the flexible rail 16 and web. The ball 26 and the socket 28 components, including the track channel 22, may be formed of stainless steel. In other embodiments, the ball 26, the socket 28, and/or the track channel 22 may be formed of a suitable rigid biocompatible polymeric material or any combination of stainless steel and polymeric materials.

The nestable ball 26 and socket 28 components are disposed such that their adjacent surfaces coact. The adjacent ball 26 and socket 28 assemblies 29 are formed such that the ball 26 may be located (e.g., pressed) into the adjacent socket 28 and is retained therein. The projections 33 formed inside the socket 28 are adapted and configured to engage and compress the surface of the ball 26. The ball 26 and the socket 28 each have a central bore such that the multiple ball 26 and socket 28 assemblies 29 form the central bore 30 to accommodate the tension wire 32 extending therethrough. The tension wire 32 is fixedly attached to the distal end of the rigidizable guide member 20 and is coupled to the rigidizing mechanism 24 (FIG. 1) at the proximal end such that the tension wire 32 can be tensioned and/or relaxed. The tension wire 32 may be fixedly attached to the distal end of the rigidizable guide member 20 in any suitable manner such that the tension wire 32 is not pulled through the central bore 30 when the rigidizing mechanism 24 tensions the tension wires 32. For example, the tension wires 32 may comprise balls welded or molded onto the ends of the tension wires 32 and fixedly attached to the distal end of the rigidizable guide member 20 to ensure the tension wires 32 cannot be pulled through the central bore 30. Alternatively, terminations may comprise knots formed in the ends of the tension wires 32, or any suitable fastener or crimp may be provided to prevent the tension wires 32 from being drawn through the central bore 30 in operation. When the tension wire 32 is relaxed, the adjacent surfaces of the ball 26 and the socket 28 can rotate relative to each other and thus the rigidizable guide member 20 is rendered flexible. In its normally flexible state, the rigidizable guide members 20a,b can move flexibly and slidably along the flexible rail 16 and track channel 22 to follow the contoured shape of the endoscope 18 (FIG. 1). When the rigidizing mechanism 24 is actuated, the tension wire 32 imparts a load that clamps the adjacent surfaces of the ball 26 and socket 28 assemblies 29 together at its current relative orientation, thereby fixing or locking the shape of the rigidizable guide member 20. When one of the rigidizable guide members 20a,b is rigid, it may be used as a guide for advancing the endoscope 18, or for advancing the other guide member 20a,b, along the track channel 22 and rail 16 further into the natural opening of the patient (e.g., the colon, esophagus, etc.). The tension wire 32 may be formed of any suitable material and in one embodiment may be formed of stainless steel.

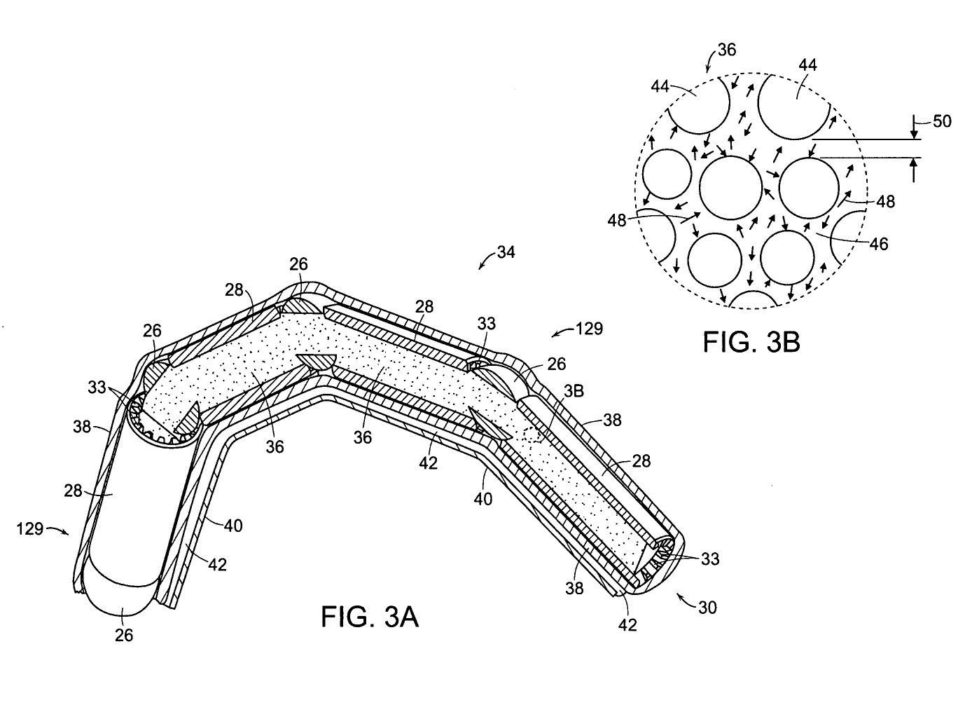

FIG. 3A illustrates a partial sectional view of one embodiment of a rigidizable guide member 34 taken along the longitudinal axis. The rigidizable guide member 34 is similar to the rigidizable guide members 20, 20a, and 20b shown in FIGS. 1 and 2. The rigidizable guide member 34 comprises a continuous length of assemblies 129 each comprising the coacting nestable ball 26 and socket 28 components with a state-change material 36 provided in the central bore 30. The socket 28 comprises the projections 33 configured to engage and compress the surface of the ball 26. The state-change material 36 may be a biocompatible material suitable to render the rigidizable guide member 34 rigid when a vacuum is applied to the central bore 30 by the rigidizing mechanism 24 (e.g., a vacuum/pump arrangement in this embodiment). To ensure an airtight seal between the coacting surfaces of the balls 26 and sockets 28 and to obtain suitable vacuum suction, a flexible membrane 38 is provided over the length of the rigidizable guide member 34. The flexible membrane 38 may be formed of any suitable flexible polymeric material, such as a suitable type of low stretch material like a polyester film, or a polymer film with some cord or fiber reinforcement. In one embodiment, the flexible membrane 38 may be formed of material similar to the flexible sheath 14 material as discussed above. In the ball 26 and socket 28 assembly 129, the socket 28 is substantially smooth and does not comprise a track channel. Rather, a track channel 40 suitable to receive the flexible rail 16 is formed on the flexible membrane 38 as a generally continuous unitary piece of material. A web 42 formed in the flexible membrane 38 material supports the track channel 40.

In one embodiment, the state-change material 36 may comprise a material that behaves as a fluid and can take the shape or form of an object and when a vacuum is applied becomes solid and rigid. The state-change material 36 may be introduced into the central bore 30 as a fluid. The state-change material 36 fills the volume defined by the central bore 30 and conforms to its the geometry. The state-change material 36 comprises hard solid bodies suspended in a liquid medium. A transition fluid creates a transition clearance between the hard solid bodies such that the state-change material 36 remains flexible. In this state, the adjacent surfaces of the balls 26 and the sockets 28 can rotate relative to each other and thus the rigidizable guide member 34 is rendered flexible and is able to flexibly and slidably move along the track channel 40 and follow the contoured shape of the endoscope 18 (FIG. 1) along the flexible rail 16. A vacuum may be applied to the state-change material 36 to withdraw the transition fluid by suction. When the transition fluid is removed, the hard solid bodies contact each other and interlock the state-change material 36. The quantity of the transition fluid may be selected such that there is no appreciable change in volume when the transition fluid is removed. In the interlocked state, the hard solid bodies are packed together tightly to form a solid rigid component within the central bore 30 and thus fixes or locks the shape of the rigidizable guide member 34 rendering it rigid. When the rigidizable guide member 34 is rigid, it may be used as a guide for advancing the endoscope 18 along the channel 40 and rail 16 further into the natural opening of the patient (e.g., the colon, esophagus, etc.). This process is completely reversible. Therefore, removing the vacuum and pumping the transition fluid back into the central bore 30 restores the clearance volume between the hard solid bodies to re-fluidize the rigid interlocked state-change material 36 and thus the rigidizable guide member 34 regains its flexibility.

FIG. 3B is an enlargement of one embodiment of a state-change material 36 that may be introduced into the central bore 30 for the purpose of rigidizing the rigidizable guide member 34. In the embodiment illustrated in FIG. 3B, the state-change material 36 is shown prior to a vacuum being applied to remove the transition fluid. In one embodiment, the state-change material 36 is a reversible state-changeable mixture comprising a plurality of hard solid bodies 44 and a carrier medium 46, with the carrier medium 46 filling any voids or interstices between the hard solid bodies 44. Within the mixture, the hard solid bodies 44 can be caused to transition from a formable state, preferably a near-liquid or fluent condition of mobility, to a stable, force-resisting condition through introduction and then extraction of a slight excess quantity of the carrier medium 46 beyond that required to fill the interstices of the hard solid bodies 44 when closely packed. In most embodiments, the carrier medium 46 is a liquid preferably excluding any air or other gases from the mixture. However, some embodiments may be use a carrier medium that is a liquid-gas froth. In one embodiment, the hard solid bodies 44 may be have a spherical form and may be surrounded by a liquid medium 46 with the same density as the bodies 44. The state-change material 36 also comprises an excess amount of liquid medium, hereinafter referred to as transition liquid 48. Pressure is applied against the hard solid bodies 46 to add a suitable quantity of transition liquid to create a small clearance volume 50. Otherwise, the hard solid bodies 44 are packed and nested against one another inside chamber the central bore 30. Therefore, the packed and abutted hard solid bodies 46 act as a solid fill in regard to their resistance to compression. The transition liquid 48 may be added to fill any added clearance volume. If the hard solid bodies 44 are of a small diameter, the added volume to allow clearance is also very small.

The state-change material 36 can be rapidly shifted from a formable (preferably near-liquid or fluent) state to a stable force-resisting state and back again to the formable state, through slightly altering the carrier-solid proportions of the state-change material 36 mixture. Embodiments are characterized by one or more of the following advantages: the ability to pressurize the state-change material 36 mixture and drive it against a surface as if it were a liquid; the ability to conform due to the negligible volumetric change that accompanies a state change; the ability to effect the state-change with a very small volume of single-constituent transfer and with consequently small actuation devices without the need for a vacuum pump, without chemical reactions, and with no need for thermal or electrical energy to be applied to the mixture; and the ability to tailor the mixture to satisfy a wide variety of physical specifications in either the flowable or the rigid stable state.

The state-change material 36 mixture can be used to fill the volume defined by the central bore 30 and is reusable. The state-change material 36 mixture can also be used in any product or shape that benefits from the incorporation of arbitrary reformability or precise reconfigurability. The state-change material 36 mixture provides useful properties for use in a supportive elements or apparatus such as the rigidizable guide member 34.

The state-change material 36 mixture in its formable state may be loosely compared to quicksand, while the state-change material 36 mixture in its stable state may resemble hard-packed sand or even cement, with the transition being caused by the transfer of a relatively small amount of liquid. Hence the state-change material 36 mixture, while in the formable state, includes enough liquid 46 to fill the interstices between the nested solid bodies 44, and an excess amount of liquid that is referred to as the transition liquid 48. In the stable state the transition liquid 48 is absent and the hard solid bodies 44 are completely packed or nested.

In one embodiment, the hard solid bodies 44 are uniform, generally ordered, and closely spaced, with the predominate mass of the hard solid bodies 44 close-packed and touching. To create mobility, the transition liquid 48 is introduced in just-sufficient quantity to create a fluent condition by providing the clearance 50 between some of the hard solid bodies 44, which clearance permits the introduction of at least two simultaneous slip planes between ordered masses of the hard solid bodies 44 at any point in the state-change material 36 mixture. The hard solid bodies 44 themselves separate freely from one another under movement of the liquid and without turbulent mixing, and shift relative to one another generally in ordered bulk masses. The hard solid bodies 44 should be of a density that is close enough to that of the liquid 46 to permit flow of the hard solid bodies 44 along with the liquid 46, or should have a size or structure that facilitates movement of the hard solid bodies 44 along with the liquid 46.

In a method according to one embodiment, the state-change material 36 mixture while in the formable state is first made to conform to the volume define by the central bore 30. The hard solid bodies 44 in the state-change material 36 mixture are then caused to transition from the fluent condition to the stable condition through extraction of the transition liquid 48. This extraction removes the clearance volume 50 required to provide slip-planes between ordered masses of the hard solid bodies 44, thereby causing the hard solid bodies 44 to make nested, packed, interlocking or otherwise stable consolidated contact. The state-change material 36 mixture, now in the stable state, has a surface that conforms to the central bore 30.

Distribution of uniform pressure against the surface of each hard solid body 44, coupled with the clearance volume 50 furnished by the transition liquid 48, assures that the hard solid bodies 48 are not forced against one another while the mixture is in the fluent condition. This elimination of body-to-body compression forces in turn prevents the bodies from sticking together and resisting displacement while the mixture is in the fluent condition. Pressure forces in the liquid 46 may be induced by a two-way pump or other transfer system.

The hard solid bodies 44 themselves may have various geometries and may be provided within the state-change material 36 mixture in one uniform type, or there may be two or more types or sizes of bodies dispersed or layered within a mixture. For example spherical bodies of one size might have smaller bodies filling the interstices between the larger bodies, or a layer of short fiber bodies might float above a layer of spherical bodies. Flake-like bodies can be also be used, in which case the flat faces of the bodies can be pressed against one another to create a force-resisting body mass. The flat faces provide many times the contact area of abutting spheres, with accordingly higher friction or adhesion potential when consolidated against one another. If the flakes are in the form of a laminate that has one side heavier than the carrier medium and one side lighter, and if the flakes are closely spaced and in a medium which suppresses turbulence and solid body tumbling, the bodies will tend to be supported in, and to be consolidated in, an ordered parallel configuration. In this case, as with the spherical bodies, the transition liquid quantity will be just sufficient to create shear motion of body masses under low displacement forces. State-change material 36 mixtures with more than one type or size of body can be used with the bodies either intermingled or layered separately, as by differing densities or the inability of bodies of one layer to pass through bodies in the adjacent layer. Bodies of different sizes or types may also be separated from one another by flexible or extensible porous materials or fabrications that allow passage of liquids but not of the confined bodies. The degree of accuracy or irregularity on the surface of a stabilized mass of the mixture may depend upon the relationship between the fineness of the bodies and the dimensions to be captured, and the size and degree of regular packing order of the solid bodies. If the bodies are very small compared to the contours of a shape that is to be replicated, or if the interstices between larger bodies in the mixture are filled by such smaller bodies, the mobile solid bodies of the mixture will consolidate and assume a near-net shape relative to any impressed shape when the transition liquid is extracted from the mixture. A more detailed description of the state-change material 36 is provided in U.S. Pat. No. 7,172,714 to Jacobson, and U.S. Pat. No. 6,780,352 to Jacobson, which are both incorporated herein by reference.

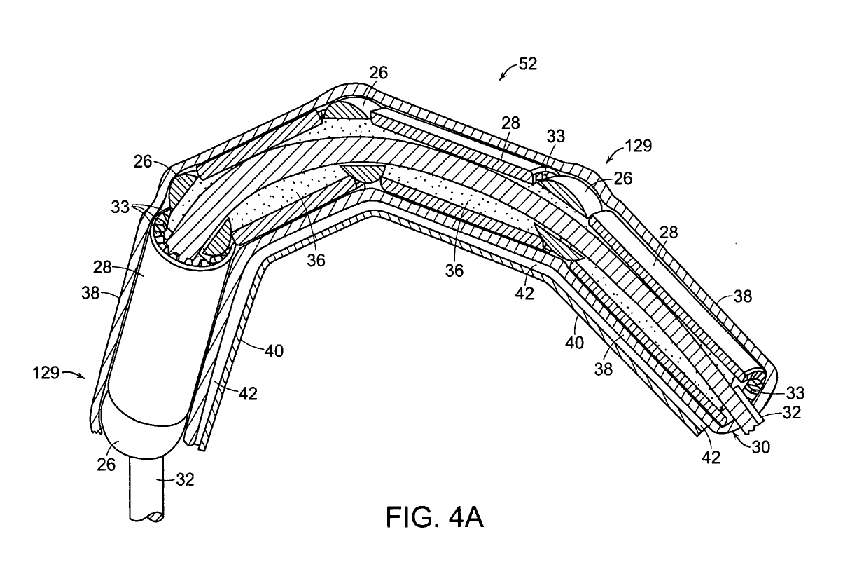

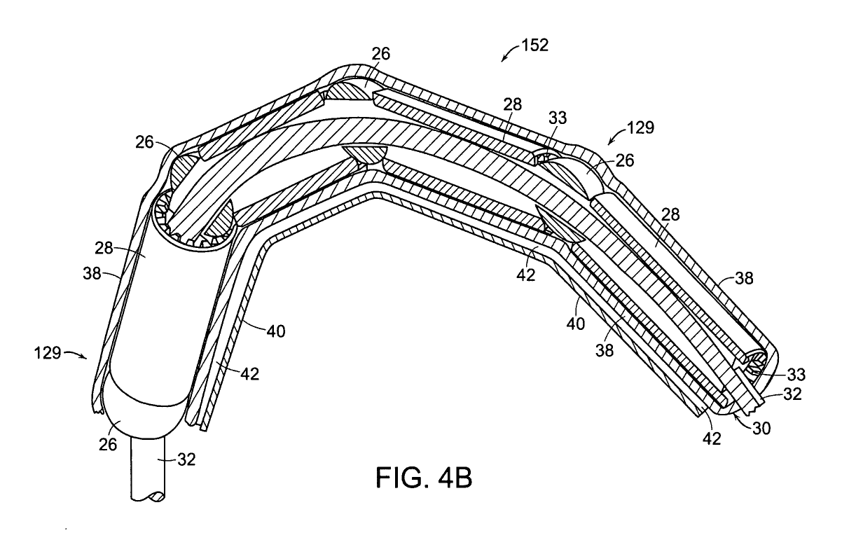

FIG. 4A illustrates a partial sectional view of one embodiment of a rigidizable guide member 52 taken along the longitudinal axis. The rigidizable guide member 52 is similar to the rigidizable guide members 20, 20a, 20b, and 34 shown in FIGS. 1, 2, and 3A. The rigidizable guide member 52 comprises a continuous length of assemblies 129 each comprising the coacting nestable ball 26 and socket 28 components. The socket 28 comprises the projections 33 configured to engage and compress the surface of the ball 26. A combination of the tension wire 32 and the state-change material 36 are provided in the central bore 30. The flexible membrane 38 is provided over the length of the rigidizable guide member 52. The flexible membrane 38 may be formed of any suitable material as previously described. In the ball 26 and socket 28 assembly 129, the socket 28 is substantially smooth and does not comprise a track channel. Rather, the track channel 40 suitable to receive the flexible rail 16 is formed on the flexible membrane 38 as a generally continuous unitary piece of material. A web 42 formed in the flexible membrane 38 material supports the track channel 40.

A vacuum generated by a portion of the rigidizing mechanism 24 may be applied to the central bore 30 via the vacuum ports 31a,b (FIG. 1) to remove the transition fluid 48 in the central bore 30 and cause the hard solid bodies 44 to be nested, packed, interlocked or otherwise rigidly stable consolidated contact. Thus, the state-change material 36 transitions state from a fluent state to a solid rigid state to fix and lock-in the shape of the rigidizable guide member 52 rendering it rigid. If additional rigidity is required, tension may be applied to the ball 26 and socket 28 assemblies 129 by tensioning the tension wire 32 with a wire tensioner portion of the rigidizing mechanism 24. Thus, in combination, the rigidizable guide member 52 may be rendered rigid such that the endoscope 18 may be advanced along the channel 40 and rail 16 into the natural opening of the patient (e.g., the colon, esophagus, etc.). Because, the process is completely reversible, removing the tension on the tension wire 32 and pumping the transition fluid 48 back into the central bore 30 re-fluidizes the packed interlocked hard solid bodies 44 (FIG. 3B) of the state-change material 36 and the rigidizable guide member 52 regains its flexibility. In its normally flexible state, the rigidizable guide member 52 may be advanced further into the natural opening of the patient.

FIG. 4B illustrates a partial sectional-view of one embodiment of a rigidizable guide member 152 taken along the longitudinal axis. The rigidizable guide member 152 is similar to the rigidizable guide members 20, 20a, 20b, 34, and 52 shown in FIGS. 1, 2, 3A, and 4A. The rigidizable guide member 152 comprises a continuous length of assemblies 129 each comprising the coacting nestable ball 26 and socket 28 components. The socket 28 comprises the projections 33 configured to engage and compress the surface of the ball 26. The tension wire 32 is provided through the central bore 30. In the embodiment illustrated in FIG. 4B, no state-change material is provided in the central bore 30. The flexible membrane 38, however, is provided over the length of the rigidizable guide member 34. The flexible membrane 38 may be formed of any suitable material as previously discussed. In the ball 26 and socket 28 assembly 129, the socket 28 is substantially smooth and does not comprise a track channel. Rather, the track channel 40 suitable to receive the flexible rail 16 is formed on the flexible membrane 38 as a generally continuous unitary piece of material. A web 42 formed in the flexible membrane 38 material supports the track channel 40.

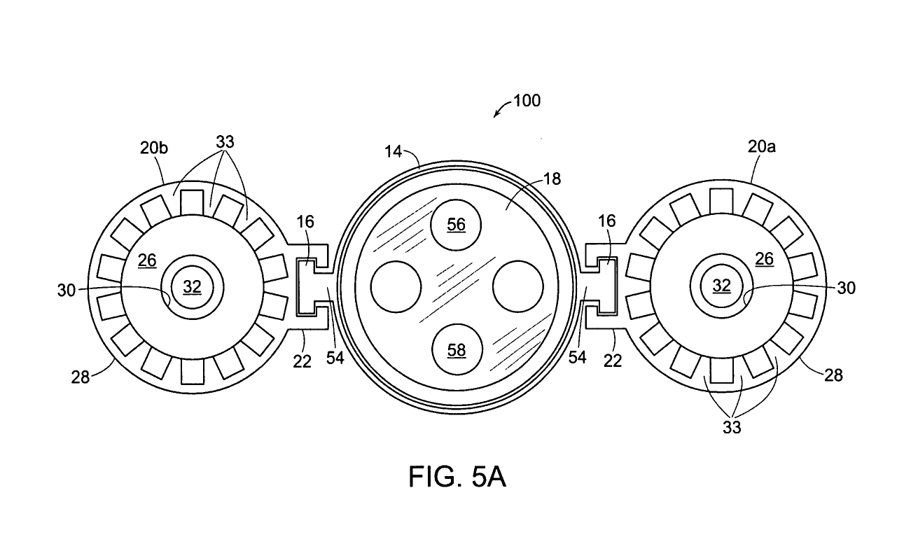

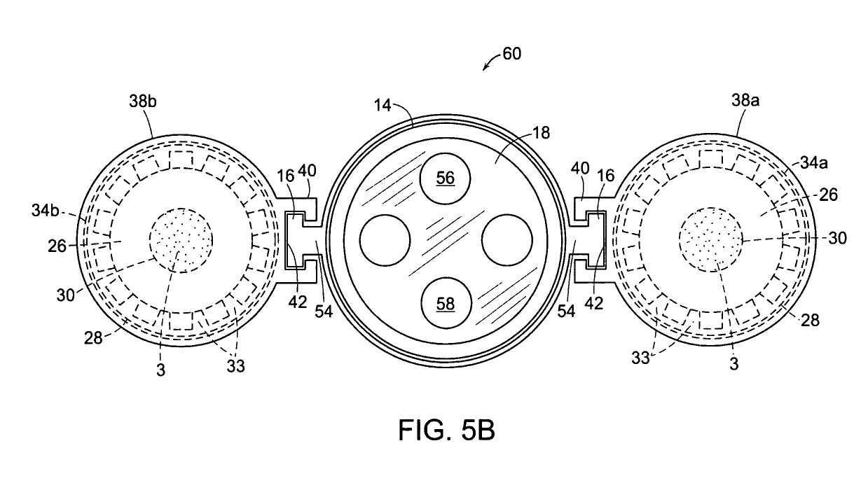

FIG. 5A is an end view of one embodiment of a medical apparatus 100. The medical apparatus 100 comprises the endoscope 18, the first rigidizable guide member 20a, the second rigidizable guide member 20b, and the flexible sheath 14 provided substantially over the entire longitudinal length of the endoscope 18. Radially, the rigidizable guide members 20a,b may be coupled to the endoscope 18 by way of an endorail type connection. In the illustrated embodiment, the endorail connection is formed of the flexible rail 16 and the corresponding track channel 22. The flexible rail 16 is disposed along the sheath 14. The flexible rail 16 comprises a rail web 54 and is supported by the flexible sheath 14. A cross-section of the flexible rail 16 and web define a general “T” configuration. The flexible rail 16 can be a generally continuous, unitary piece of material which extends longitudinally along the length of the flexible sheath 14. The rigidizable guide members 20a,b are positioned along a longitudinal axis of the endoscope 18. The first rigidizable guide member 20a and the second rigidizable guide member 20b comprises a track channel 22 to slideably receive the flexible rail 16. The track channel 22 is supported by the rigidizable guide members 20a,b. The end portion of the socket 28 also may comprise the projections 33 configured to engage and compress the ball 26 component. A cross-section of the track channel 22 defines a general “C” configuration. Each rigidizable guide member 20a,b may be advanced or retracted independently of each over a length of the adjustable portion (i.e., the flexible, steerable articulating section) of the endoscope 18. This adjustable portion of the endoscope 18 is usually the distal five or six inch portion of the endoscope 18. The endoscope 18 comprises a viewing element 56 and one or more working channels 58. The endoscope 18 may be steered using two or more wires using generally well known techniques.

Each rigidizable guide members 20a,b also comprises the central bore 30 defining a channel. The tension wire 32 is disposed in the central bore 30. The tension wire 32 is employed to render the rigidizable guide members 20a,b rigid and prevent them from flexing or bending upon the application of a rigidizing force. Each of the tension wires 32 is fixedly attached to the distal end of the rigidizable guide members 20a,b in any suitable manner such that the tension wire 32 is not pulled through the central bore 30 when tensioning the tension wires 32 as previously discussed. The tension wire 32 in each rigidizable guide member 20a,b may be operated independently of each other such that one rigidizable guide member 20a may be in a rigid state while the other rigidizable guide member 20b remains in a flexible state. Flexibility is restored when the tensioning force is removed. The process may be repeated as necessary. In one embodiment, when activated, the tension wires 32 apply a clamping force on the rigidizable guide members 20a,b to render them rigid or firm and difficult to bend or flex. When the tensioning force is released, the rigidizable guide members 20a,b return to their normally flexible state. The tension wires 32 may be actuated by a wire tensioner or other rigidizing mechanism 24.

Embodiments of rigidizable guide members may be formed in various shapes, sizes, and materials. In one embodiment, rigidizable guide members may be formed with helical wires (e.g., coil spring). A highly flexible sheath may be provided over the rigidizable guide members. A central bore through the rigidizable guide members may be filled with biocompatible state-change material 36 to render the rigidizable guide member rigid when a vacuum is applied to the central bore. In another embodiment, rigidizable guide members may be formed by connecting multiple cylindrical elements held together with a highly flexible sheath. The cylindrical elements provide radial stiffness. The central bore may be filled with a combination of the state-change material 36 and the rigidizing may be assisted by employing one or more tension wires 32.

FIG. 5B is an end view of one embodiment of a medical apparatus 60. The medical apparatus 60 comprises the endoscope 18, a first rigidizable guide member 34a covered with a first flexible membrane 38a, a second rigidizable guide member 34b covered with a second flexible membrane 38b, the flexible sheath 14 provided substantially over the entire longitudinal length of the endoscope 18. The first and second rigidizable guide members 34a,b are similar to the rigidizable guide member 34 shown in FIG. 3A. The state-change material 36 is provided in the central bore 30. The first and second flexible membranes 38a,b are similar to the flexible membrane shown in FIG. 3A. To ensure an airtight seal between the coacting surfaces of the ball 26 (FIG. 3A, for example) and socket 28 assemblies and to obtain suitable vacuum suction, the flexible membranes 38a,b are provided over the length of and over the distal end of each respective first and second rigidizable guide members 34a,b. As previously described, the flexible membranes 38a,b may be formed of any suitable flexible polymeric material, such as a suitable type of low stretch material like a polyester film, or a polymer film with some cord or fiber reinforcement. The track channel 40 is suitable to receive the flexible rail 16 and is formed integrally with the flexible membranes 38a,b as a generally continuous unitary piece of material. The web 42 is formed on the flexible membranes 38a,b supports the track channel 40. The end socket 28 also may comprise the projections 33 configured to engage the surface of the ball 26.

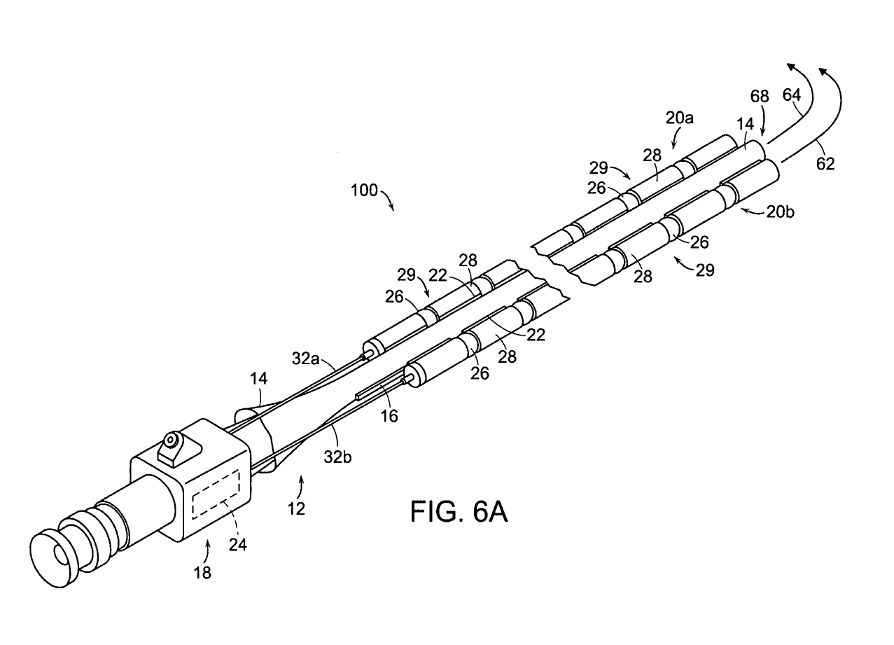

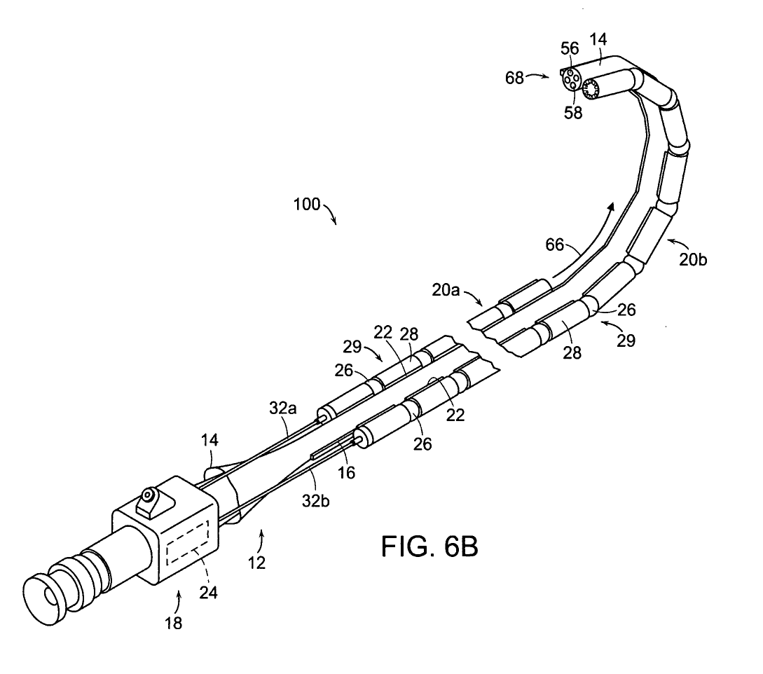

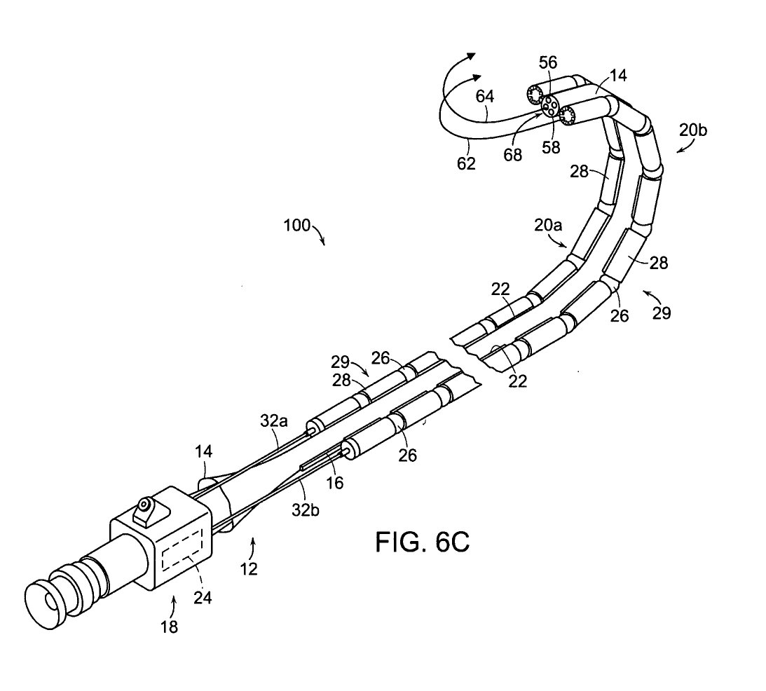

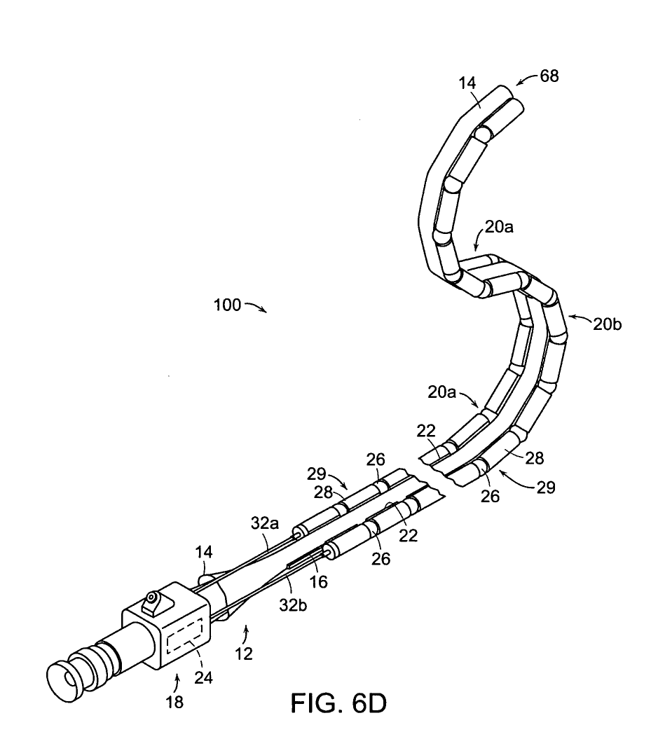

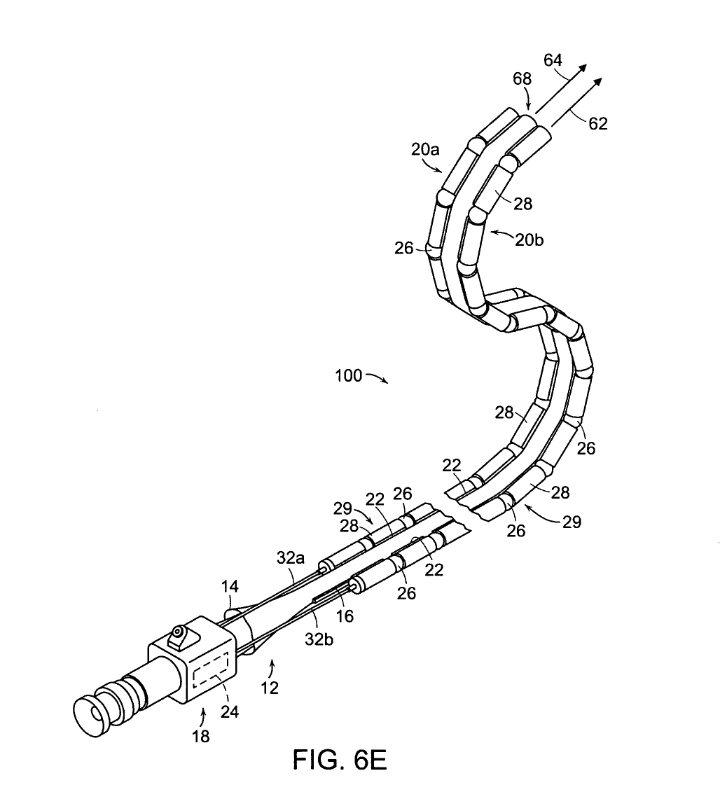

FIGS. 6A-E illustrate one embodiment of a method of employing the medical apparatus 100 comprising the first and second rigidizable guide members 20a,b to advance and maneuver the endoscope 18 into a natural hollow body organ of a patient having a tortuous and unsupported anatomy, such a colon. FIG. 6A illustrates one embodiment of the medical apparatus 100 comprising the endoscope 18 and the first and second rigidizable guide members 20a,b. A steerable distal tip 68 of the endoscope 18 is positioned by means of endoscope cables such that the endoscope 18 is aligned with first and second rigidizable guide members 20a,b. Tension is applied to the first tension wire 32a by a rigidizing mechanism 24 located in the handle of the endoscope 18. This renders the first rigidizable guide member 20a rigid and forms a rigid guide for the endoscope 18 to follow. The endoscope 18 is now advanced on track 22 and rail 16 (e.g., endorail) along the rigid guide path formed by the rigid first rigidizable guide member 20a. The endoscope 18 may advance in an arcuate path in the direction indicated by arrow 64 by a distance of several inches, such as 5-6 inches, for example, that is substantially the length of the steerable portion of the endoscope 18. Once the endoscope 18 has been advanced, tension on the second rigidizable guide member 20b is released (e.g., relaxed) and it is advanced along the track 22 and rail 16 in an arcuate path in the direction indicated by arrow 62 to the distal tip 68 of the endoscope 18 and is placed into position with the endoscope 18.

FIG. 6B illustrates one embodiment of the medical apparatus 100 comprising the endoscope 18 and the first and second rigidizable guide members 20a,b with the endoscope 18 and the second rigidizable guide member 20b in the advanced position as described with reference to FIG. 6A. The distal tip 68 of the endoscope 18 is substantially aligned with the second rigidizable guide member 20b. Tension is now applied to the second tension wire 32b by the rigidizing mechanism 24 and tension is released (e.g., relaxed) from the first rigidizable guide member 20a. This renders the second rigidizable guide member 20b rigid and restores flexibility to the first rigidizable guide member 20a. The second rigidizable guide member 20b now forms a rigid guide path for the first rigidizable guide member 20a to follow as it advances in the direction indicated by arrow 66 to the distal tip 68 of the endoscope 18 until the first and second rigidizable guide members 20a,b and the distal tip 68 of the endoscope 18 are substantially aligned.

FIG. 6C illustrates one embodiment of the medical apparatus 100 comprising the endoscope 18 and the first and second rigidizable guide members 20a,b with the endoscope 18 and the first and second rigidizable guide members 20a,b in the advanced position as described with reference to FIG. 6B. The distal tip 6.8 of the endoscope 18 is substantially aligned with the first and second rigidizable guide members 20a,b. Tension is now applied to the first tension wire 32a by the rigidizing mechanism 24 and tension is released from the second rigidizable guide member 20b. This renders the first rigidizable guide member 20a rigid and restores flexibility to the second rigidizable guide member 20b. The first rigidizable guide member 20a now forms a rigid guide path for the endoscope 18 to follow as it advances in an arcuate path in the direction indicated by arrow 64 by a distance of several inches. Once the endoscope 18 has been advanced, the second rigidizable guide member 20b is relaxed and is advanced along the track 22 and rail 16 in an arcuate path in the direction indicated by arrow 62 to the distal tip 68 of the endoscope 18 and is placed into position with the endoscope 18.

FIG. 6D illustrates one embodiment of the medical apparatus 100 comprising the endoscope 18 and the first and second rigidizable guide members 20a,b with the endoscope 18 and the second rigidizable guide member 20b in the advanced position as described with reference to FIG. 6C. The distal tip 68 of the endoscope 18 is substantially aligned with the second rigidizable guide member 20b. Tension is now applied to the second tension wire 32b by the rigidizing mechanism 24 and tension is released (e.g., relaxed) from the first rigidizable guide member 20a. This renders the second rigidizable guide member 20b rigid and restores flexibility to the first rigidizable guide member 20a. The second rigidizable guide member 20b now forms a rigid guide path for the first rigidizable guide member 20a to follow as it advances to the distal tip 68 of the endoscope 18 until the first and second rigidizable guide members 20a,b and the distal tip 68 of the endoscope 18 are substantially aligned.

FIG. 6E illustrates one embodiment of the medical apparatus 100 comprising the endoscope 18 and the first and second rigidizable guide members 20a,b with the endoscope 18 and the first and second rigidizable guide members 20a,b in the advanced position as described with reference to FIG. 6B. The distal tip 68 of the endoscope 18 is substantially aligned with the first and second rigidizable guide members 20a,b. Tension is now applied to the first tension wire 32a by the rigidizing mechanism 24 and tension is released (e.g., relaxed) from the second normally rigidizable guide member 20b. This renders the first rigidizable guide member 20a rigid and restores flexibility to the second rigidizable guide member 20b. The first rigidizable guide member 20a now forms a rigid guide path for the endoscope 18 to follow as it advances in the direction indicated by arrow 64 by a distance of several inches. Once the endoscope 18 has been advanced, the second rigidizable guide member 20b is advanced along the track 22 and rail 16 in the direction indicated by arrow 62 to the distal tip 68 of the endoscope 18 and is placed into position with the endoscope 18.

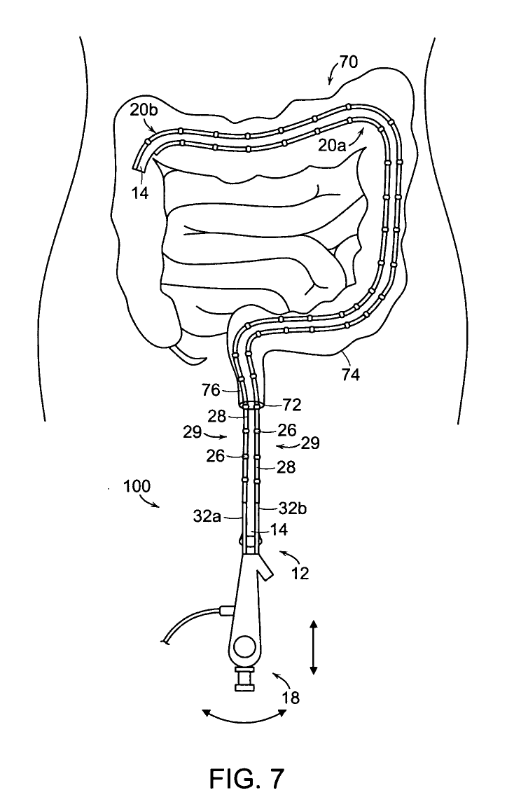

FIG. 7 shows one embodiment of the medical apparatus 100 inserted into a hollow body organ or a natural opening of a patient. The medical apparatus 100 is inserted into the colon 70 through the anus 72. The colon 70 includes a sphincter muscle 74 disposed between the anus 72 and the rectum 76. The medical apparatus 100 is maneuvered through several turns through the colon 70 by employing the procedure outlined with reference to FIGS. 6A-E. The procedure may be repeated as necessary until the endoscope 18 is located in the desired position within the natural opening of the patient e.g., the colon 70. Also, a procedure similar to the procedure outlined with reference to FIGS. 6A-E may be employed for the first and second rigidizable guide members 34a,b comprising the state-change material 36 in the central bore 30. In such embodiment, a vacuum and pump mechanism may be employed to rigidize and restore flexibility to the first and second rigidizable guide members 34a,b. For example, as previously discussed, a vacuum may be applied to the central bore 30 to withdraw the transition liquid 48 to render the rigidizable guide members 34a,b rigid. A pump may then be employed to pump the transition liquid 48 back into the central bore 30 to restore the flexibility to the rigidizable guide members 34a,b. Likewise similar procedures may be applied to the rigidizable guide members 54a,b.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the various embodiments of the invention described herein will be processed before surgery. First, a new or used instrument is obtained and if necessary cleaned. The instrument can then be sterilized. In one sterilization technique, the instrument is placed in a closed and sealed container, such as a plastic or TYVEK bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

It is preferred that the device is sterilized. This can be done by any number of ways known to those skilled in the art including beta or gamma radiation, ethylene oxide, steam.

Although the various embodiments of the invention have been described herein in connection with certain disclosed embodiments, many modifications and variations to those embodiments may be implemented. For example, different types of end effectors may be employed. Also, where materials are disclosed for certain components, other materials may be used. The foregoing description and following claims are intended to cover all such modification and variations.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A medical apparatus, comprising:
a flexible sheath adapted to receive an endoscope;
a first flexible rail formed on the flexible sheath, the first flexible rail extending longitudinally along the length of the flexible sheath; and
a first rigidizable guide member comprising a first track channel adapted to receive the first flexible rail, the first rigidizable guide member is adapted to slideably move along the first flexible rail and the first track channel.

2. The medical apparatus of claim 1, comprising:
a first central bore extending through the first rigidizable guide member; and
a first rigidizing component disposed in the first central bore;
wherein the first rigidizable guide member is rendered rigid when the first rigidizing component is actuated; and
wherein the first rigidizable guide member is rendered flexible when the first rigidizing component is deactuated.

3. The medical apparatus of claim 2, comprising:
a rigidizing mechanism coupled to the first rigidizable guide member, wherein the first rigidizable guide member is rendered inflexible when the rigidizing mechanism actuates the first rigidizing component and the first rigidizable guide member is rendered flexible when the rigidizing mechanism deactuates the first rigidizing component.

4. The medical apparatus of claim 2, wherein the first rigidizing component comprises a tensioning wire to apply a tensioning force to rigidize the first rigidizable guide member.

5. The medical apparatus of claim 2, wherein the first rigidizing component comprises:
a state-change material to rigidize the first rigidizable guide member when the state-change material is in a rigid state.

6. The medical apparatus of claim 5, comprising:
a first flexible membrane disposed over the first rigidizable guide member.

7. The medical apparatus of claim 1, wherein the first rigidizable guide member comprises:
a socket; and
a ball partially inserted in the socket;
wherein adjacent surfaces of the ball and the socket coact and can rotate relative to each other in a flexible state; and
wherein the adjacent surfaces of the ball and socket are locked in place when a tensioning force is applied to the ball and the socket.

8. The medical apparatus of claim 1, comprising:
a second flexible rail formed on the flexible sheath, the second flexible rail extending longitudinally along the length of the flexible sheath; and
a second rigidizable guide member comprising a second track channel adapted to receive the second flexible rail, the second rigidizable guide member is adapted to slideably move along the second flexible rail and the second track channel.

9. The medical apparatus of claim 8, comprising:
a second central bore extending through the second rigidizable guide member; and
a second rigidizing component disposed in the second central bore;
wherein the second rigidizable guide member is rendered rigid when the second rigidizing component is actuated; and
wherein the second rigidizable guide member is rendered flexible when the second rigidizing component is deactuated.

10. The medical apparatus of claim 9, wherein the rigidizing mechanism is coupled to the second rigidizable guide member, and wherein the second rigidizable guide member is rendered inflexible when the rigidizing mechanism actuates the second rigidizing component and the second rigidizable guide member is rendered flexible when the rigidizing mechanism deactuates the second rigidizing component.

11. The medical apparatus of claim 9, wherein the rigidizing component comprises a second tensioning wire to apply a tensioning force to rigidize the second rigidizable guide member.

12. The medical apparatus of claim 9, wherein the second rigidizing component comprises:
a state-change material to rigidize the second rigidizing component when the state-change material is in a rigid state.

13. The medical apparatus of claim 12, comprising:
a second flexible membrane disposed over the second rigidizable guide member.

14. The medical apparatus of claim 8, wherein the first and the second rigidizable guide members are independently positionable along the longitudinal length of the flexible sheath.

15. A method of maneuvering a medical apparatus in a natural hollow body organ, the medical apparatus comprising a flexible sheath adapted to receive an endoscope; a first and second flexible rail formed on the flexible sheath, the first and second flexible rails extending longitudinally along the length of the flexible sheath; and a first and second rigidizable guide members comprising respective first and second track channels adapted to receive the respective first and second flexible rails, the first and second rigidizable guide members are adapted to independently slideably move along the first and second flexible rails and the first and second track channels, the method comprising:
providing an endoscope in the flexible sheath;
rigidizing the first rigidizable guide member;
relaxing the second rigidizable guide member;
advancing the endoscope along the first flexible rail into the natural hollow body organ; and
advancing the second rigidizable guide member along the second flexible rail into the natural hollow body organ.

16. The method of claim 15, comprising:
positioning the second rigidizable guide member with the endoscope.

17. The method of claim 15, comprising:
rigidizing the second rigidizable guide member;
relaxing the first rigidizable guide member; and
advancing the first rigidizable guide member along the second flexible rail into the natural hollow body organ.

18. The method of claim 17, comprising:
positioning the first rigidizable guide member with the endoscope.

19. A method comprising:
obtaining a medical apparatus, wherein the medical apparatus comprises:
a flexible sheath adapted to receive an endoscope;
a first flexible rail formed on the flexible sheath, the first flexible rail extending longitudinally along the length of the flexible sheath; and
a first rigidizable guide member comprising a first track channel adapted to receive the first flexible rail, the first rigidizable guide member is adapted to slideably move along the first flexible rail and the first track channel;
sterilizing the surgical instrument; and
storing the surgical instrument in a sterile container.
